(19) United States
(12) Patent Application Publication
(10) Pub. No.: US 2005/0013847 A1
(43) Pub. Date: Jan. 20, 2005

(54) DELIVERY SYSTEMS OF HOMOGENEOUS, THERMOREVERSIBLE ALGINATE FILMS

(75) Inventors: Arthur Ballard, Westport, ME (US); Christopher Sewall, Hope, ME (US); James Modliszewski, Brick, NJ (US); William Blakemore, Topsham, ME (US); Peter Riley, Yardley, PA (US)

Correspondence Address: CONNOLLY BOVE LODGE & HUTZ, LLP, P O BOX 2207, WILMINGTON, DE, 19899, US

(73) Assignee: FMC Corporation, Philadelphia, PA (US)

(21) Appl. No.: 10/824,957

(22) Filed: Apr. 14, 2004

Related U.S. Application Data
(60) Provisional application No. 60/462,758, filed on Apr. 14, 2003.
(60) Provisional application No. 60/462,721, filed on Apr. 14, 2003.
(60) Provisional application No. 60/462,617, filed on Apr. 14, 2003.
(60) Provisional application No. 60/462,785, filed on Apr. 14, 2003.
(60) Provisional application No. 60/462,783, filed on Apr. 14, 2003.
(60) Provisional application No. 60/462,794, filed on Apr. 14, 2003.
(60) Provisional application No. 60/462,792, filed on Apr. 14, 2003.
(60) Provisional application No. 60/462,793, filed on Apr. 14, 2003.

(51) Int. Cl. A61K 31/736; A61K 47/00

(52) U.S. Cl. 424/439; 514/54

(57) ABSTRACT

Abstract of the Disclosure

The present invention is directed to a delivery system comprising a homogenous, thermoreversible gel film, wherein the gel film comprises: (i) a film forming amount of water soluble thermoreversible alginate and optionally at least one of a plasticizer, a second film former, a bulking agent, and a pH controlling agent; and (ii) an active substance. The present invention is also directed to a process for the manufacture thereof.

### Detailed Description of the Invention

### RELATED APPLICATIONS

This application claims the benefit of U.S. Provisional Application No. 60/462,793, filed on April 14, 2003.

### FIELD OF THE INVENTION

The present invention is directed to a delivery system comprising a homogenous, thermoreversible gel film, wherein the gel film comprises: (i) a film forming amount of water soluble thermoreversible alginate and optionally at least one of a plasticizer, a second film former, a bulking agent, and a pH controlling agent; and (ii) an active substance. The present invention is also directed to a process for the manufacture thereof.

### BACKGROUND OF THE INVENTION

Use of delivery systems, for example, for oral care actives in breath and cleansing strips, has increased over recent years. The present invention generally relates to delivery systems comprising a gel film that can be used to contain and deliver a wide variety of active substances.

For example, WO 02/43657 discloses the use of pullulan free edible film compositions containing at least one film forming agent, at least one bulk filler agent, at least one plasticizer. These films can contain a number of materials such as medicaments and can be used for a number of applications in oral care; e.g., breath freshening strips. The film forming agent is widely described as being any of cellulose ethers (stated to include: methylcellulose, ethylcellulose, hydroxymethylcellulose, hydroxyethylcellulose, hydroxypropylmethylcellulose; carboxymethylcellulose and derivatives thereof); modified starches (stated to include acid and enzyme hydrolyzed corn and potato starches); natural gums (stated to include gum Arabic, guar gum, locust bean gum, carrageenan gum, acacia, karaya, ghatti, tragacanth agar, tamrind gum, xanthan gum and derivatives thereof); edible polymers (stated to include microcrystalline cellulose, cellulose ethers, xanthan and derivatives thereof); hydrocolloid flours (stated to include guar gum, locust bean, microcrystalline cellulose, tara and derivatives thereof); seaweed extracts (stated to include alginate, carrageenan and derivatives thereof); and land plant extract (stated to include konjac, pectin, arabinoglactan and derivatives thereof).

WO 00/18365 discloses fast dissolving orally consumable films used to deliver breath deodorizing agents, antimicrobial agents and salivary stimulants to the oral cavity. The film forming agent to be used in making the film of the invention is said to be selected from pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, Arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, casein and mixtures thereof. These films are said to overcome the challenge of maintaining the essential oil interaction and relatively high oil content in certain films sold for oral care purposes.

### SUMMARY OF THE INVENTION

As a first embodiment, the present invention is directed to a delivery system comprising a homogenous, thermoreversible gel film, wherein said gel film comprises: (i) a film forming amount of a water soluble thermoreversible alginate and optionally at least one of a plasticizer, a second film former, a bulking agent, and a pH controlling agent; and (ii) an active substance.

As a second embodiment, the present invention is directed to a process for preparing the homogeneous gel film delivery system described immediately above comprising the steps of: (i) heating, hydrating, mixing, solubilizing, and, optionally, de-aerating the alginate and optionally at least one of a plasticizer, a second film former, a bulking agent, and a pH controlling agent in an apparatus providing sufficient shear, temperature and residence time to form a homogeneous molten composition, wherein the temperature is at or above the solubilizing temperature of the composition; (ii)adding an effective amount of an active substance either prior to or after formation of the molten composition; and (iii) cooling the molten composition containing the active substance at or below its gelling temperature to form the gel films containing the active substance.

### BRIEF DESCRIPTION OF THE DRAWINGS

Figure 1 is a partially broken away, side elevational view of the fluid mixing apparatus for mixing first and second fluids with steam that can be used in the process of the present invention.

### DETAILED DESCRIPTION OF THE INVENTION

Alginates, derived from, inter alia, brown seaweeds (Phaeophyceae sp.) are linear unbranched chemical polymers containing (1-4)-linked β-D-mannuronic acid (M) and α-L-guluronic acid (G) residues. Alginates are not random copolymers, but consist of blocks of similar and alternating residues, for example, MMMM; GGGG, and GMGM, and are generally useful in the form of alginic acid or salts thereof.

Alginates that are useful in the present invention include the monovalent salts of alginate such as sodium and potassium alginate, as well as the esterified forms of alginate such as propylene glycol alginate. All such esterified forms are encompassed within the definition of alginates as used herein. Additional examples of alginates useable in the present invention include magnesium alginate and 3-ethanol amine alginate. Such alginates may be used solely or in combination with other alginates of the present invention, and such other alginates may include small amounts of other cations provided such do not, as discussed in more detail below, adversely impact the gel formation or homogeneity of the gel film. The alginates may be fully, partially or not clarified from the raw material.

Certain alginates, e.g., sodium alginate, are generally considered to produce thermally irreversible gels with calcium ions. To the inventors' surprise, such alginates, in accordance with the present invention, have been found to produce homogeneous, thermoreversible gel films having significant film strength. In addition, certain alginates, such as propylene glycol alginate, are generally considered to be non-gelling. Also to the inventors' surprise, propylene glycol alginate has been shown to provide a homogenous, thermoreversible gel film having significant film strength.

Alginate is known to react and crosslink with available polyvalent cations (ionic) such as calcium. This crosslinking can adversely impact the film formation and thermoreversibility of the alginate gel films depending on the polyvalent cation used; e.g., magnesium does not adversely impact the thermoreversibility of the alginate gel film. Therefore, it is important that the alginate have levels of those polyvalent cations that adversely impact the film formation and thermoreversibility of the alginate film below the point that such impair film formation or thermoreversibility within the alginate system. Such polyvalent cation (e.g., calcium) levels are generally used in an amount of no higher than 5.0%, more preferably, less than 2.0%, more preferably, less than 1.0% by weight based on the dry weight of the alginate in the gel film, but this can vary depending on the effect of other components, alginate type and source, and the use of sequestering agents, etc. Sequestering or chelating agents could be added in sufficient amounts to minimize the above adverse polyvalent cation solubility (and participating activity) of e.g., calcium.

The alginate is used in the present invention in a film forming amount (e.g., an amount that adds strength to the gel film) which is distinguished from trace amounts of alginate that do not add film properties to the film. Thus, for example, in a gel film of the present invention, a film forming amount of alginate is an amount that adds film strength to the overall film. Such film forming amounts are generally at least 0.25% by weight of the dry gel film, particularly, 0.5% to 90%, more particularly, 0.5% to 50%, more particularly, 0.25 to 25% by weight of the dry gel film depending on the application.

The gel films of the present invention are homogeneous, thermoreversible films. As used herein, "homogeneous film" defines films that, to the naked eye, are visually uniform and free of defects such as lumps, cracks, particles that are undissolved that should be dissolved, non-uniform distribution of insoluble particles, etc. "Fish eyes" (mixed liquid and solid states) or "gel balls" (non-uniform gel structure) would not meet the definition of "homogeneous" as used herein.

The gel films of the present invention are homogeneous, thermoreversible gel films.

As used herein, "thermoreversible film" defines a film that has a melting temperature. As used herein the melting temperature is the temperature or temperature range over which the film softens or flows.

As used herein, "gel films" refer to thin membranes formed of structured alginate. The gel forming composition is characterized by a gel temperature, the temperature below which the molten mass must be cooled to form a self supporting structure. Optionally, a molten mass can be cast hot and allowed to cool, as well as dry to further concentrate the solids (controlled moisture removal) until a gel film is formed by the gel composition. The melt temperature of a thermoreversible gel film is higher than its gel temperature.

The gel film of the present invention includes an active substance. Examples of active substances that may be contained within the gel film is at least one of an oral care agent, a breath freshening agent, a pharmaceutical agent, a nutraceutical agent, a salivary stimulant agent, a vitamin, a mineral, a coloring agent, cosmetic ingredient, agricultural active, a sweetener, a flavorant, a fragrance or a food.

The homogeneous, thermoreversible gel film of the present invention can optionally contain at least one of a plasticizer, a second film former, a bulking agent and a pH controlling agent. The components to be added to the gel film and their amounts can vary depending on the desired characteristics of the alginate delivery system.

Examples of such a plasticizer include polyols such as glycerin, sorbitol, maltitol, lactitol, corn starch, polydextrose, fructose, solubilized oils, and polyalkylene glycols such as polyethylene glycol and propylene glycol. Such plasticizers should generally be used in an amount of at least 5%, more preferably, at least 10%, more preferably, at least 20%, more preferably, at least 30% by weight of all the components including water in the dry film depending on the desired elasticity of the delivery system. It is possible that the delivery system contains no plasticizer at all.

Examples of the second film former that can be used in the present invention include at least one of a starch, starch hydrozylate, starch derivative, cellulose gum, hydrocolloid, an alkylcellulose ether or a modified alkyl cellulose ether. Examples of the hydrocolloid include at least one of kappa carrageenan, kappa-2 carrageenan, iota carrageenan, polymannan gums such as locust bean gum or guar gums including low viscosity guar gum, dextrans, pullulan, gellan (including high and low-acyl gellan), pectin and less than fully modified versions thereof and combinations thereof. As used herein, kappa-2 carrageenan has a molar ratio of 3:6-anhydrogalactose-2-sulfate (3:6AG-2S) to 3:6-anydroglactose (3:6AG) content of 25 to 50%, iota carrageenan has a molar ratio of 3:6AG-2S to 3:6AG content of 80 to 100% and kappa carrageenan has a molar ratio of 3:6AG-2S to 3:6AG content less than that for kappa-2 carrageenan. For example, kappa carrageenan from Eucheuma cottonii, a commonly known and used seaweed source for kappa carrageenan, has a molar ratio of 3:6AG2S to 3:6AG content of less than about 10%; and iota carrageenan from Eucheuma Spinosum, a commonly known and used seaweed source for iota carrageenan, has a molar ratio of 3:6AG2S to 3:6AG content greater than about 85%. Kappa-2 carrageenan can be obtained from, for example, from Gigartina skottsbergii. Kappa, kappa-2, and iota carrageenans are different from each other in both structure and functionality. If desired, where iota, kappa or kappa-2 carrageenan is used as the second film former, such carrageenan can have a viscosity of 19 cps or less, more particularly less than 10 cps, at 75 °C in a 0.10 molar sodium chloride solution containing 1.5% of the reduced molecular weight carrageenan based on the total weight of the solution. This viscosity test can be performed using a Brookfield LVF (Brookfield Engineering Laboratories, Inc.) viscometer using Spindle #1 at 60 r.p.m. and determining the viscosity after six revolutions. An example of an alkylcellulose ether that can be used in the present invention is hydroxyethylcellulose. Examples of modified alkylcellulose ethers that can be used in the present invention include hydroxypropylcellulose and hydroxypropylmethyl-cellulose. Note that some second film formers, such as carrageenan, can contain cations that can have positive and negative impacts on gelling properties and film strength with the carrageenan and/or the alginate. Such beneficial cations include postassium, magnesium and ammonium. These cations can be present in the carrageenan or added to it from other organic or inorganic sources. These beneficial cations can be present in an amount of less than 20% by dry weight of the alginate in the gel film (including water). This amount can be varied depending on the components in the system and desired melt and sealing temperatures.

Other cations, such as calcium (as mentioned above), aluminum and chromium can adversely crosslink with alginate and/or impact carrageenan stability and should be kept to a minimum, such as less than 5%, less than 2%, less than 1% by dry weight of the alginate in the dry gel film (including water).

The alginate can be the only film former in the gel film. When the gel films of the present invention contain second film formers, the alginate can be present in an amount of at least 10%, at least 40%, at least 60% or at least 80% by weight of the total amount of film formers in the dry gel film.

Examples of the bulking agent include microcrystalline cellulose, microcrystalline starch, modified and unmodified starch, starch derivatives, inulin, starch hydrozylates, sugar, corn syrup and polydextrose. As used herein and in the claims, the term "modified starches" includes such starches as hydroxypropylated starches, acid thinned starches and the like. Examples of modified starches that can be used in the present invention include Pure Cote™ B760, B790, B793, B795, M250 and M180, Pure-Dent™ B890 and Pure-Set™ B965, all available from Grain Processing Corporation of Muscatine, Iowa, and C AraTex™ 75701, available from Cerestar, Inc. Examples of starch hydrozylates include maltodextrin also known as dextrin. Unmodified starches such as potato starch can also contribute to the film strength when combined with the hydrocolloids within the scope of the invention. In general, modified starches are products prepared by the treatment of starches, for example, acid treatment starches, enzyme treated starches, oxidized starches, cross-bonding starches and other starch derivatives. It is preferred that the modified starches be derivatized wherein side chains are modified with hydrophilic or hydrophobic groups to thereby form a more complicated structure with a strong interaction between side chains.

The amount of the bulking agent to be used in the present invention is generally 0 to 20% by weight of the dry film, but more can be used, if desired, for example, at least 20%, more preferably, at least 30% by weight of the dry film depending on the use of the delivery system.Note that starch, starch derivatives and starch hydrozylates can be multifunctional. That is, in addition to being used as bulking agents, they can be used as second film formers. When such are used as bulking agents and second film formers, they are generally used in an amount of at least 10%, preferably, at least 20%, by weight of the dry gel film.

Examples of the pH controlling agent to be used in the present invention include bases such as hydroxides, carbonates, citrates and phosphates. The pH controlling agent can be used to improve the stability of the gel film. For some compositions containing second film formers, the pH controlling agent can be chosen as the source of added beneficial cations such as potassium or ammonium. The amount of the pH controlling agent is generally in the amount of 0 to 4%, more preferably, 0 to 2% by weight of the dry film.

The dry films (e.g., 80% solids or higher) made from the gels of the present invention have been found to have, for example, a break force strength of at least 250 grams, at least 1,000 grams, at least 2,500 grams, at least 4,000 grams, at least 5,000 grams, and at least 6,000 grams, as determined using a Texture Analyzer TA 108S Mini Film Test Rig. Furthermore, dry films of the invention have been shown to have a break force strength in excess of 7,000 and 8,000 grams.

The gel films of the present invention have been found to have a solids content of at least 50%, at least 60%, at least 70%, at least 80% and at least 90% of all components in the gel film. It is understood that up to 15%, 10% or 5% water may remain strongly associated with the solids in the dry gel film.

The gel films of the invention can also contain colorants and flavorants such as sugar, corn syrup, fructose, sucrose, etc, whether or not other components, such as plasticizers, bulking agents, second film formers, etc. are present. One embodiment of a gel film of the invention comprises the alginate described herein, flavorant and water in a high solids system; e.g., greater than 50%, 60%, 65%, 75%, 80%, 85%, 90% solids.

It is possible that the gel films of the present invention can contain non-thermoreversible gums. However, so as not to adversely impact the homogeneous and thermoreversible nature of the gel films of the present invention, such non-thermoreversible films should be present in an amount of less than 50% by weight of the alginate, preferably, less than 40%, more preferably, less than 30%. Examples of such nonthermoreversible gums include (e.g., crosslinked and partially crosslinked) gums such as calcium set (crosslinked) pectins or alginates. Calcium reactive alginates and pectins, as well as their less refined forms, are considered as thermoreversible gums in the absence of divalent cations.

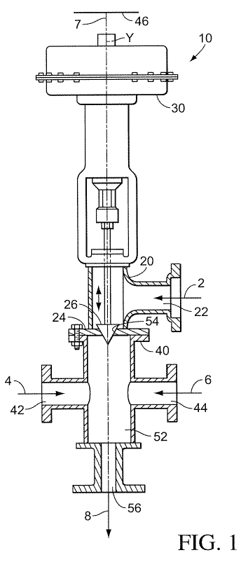

The films of the present invention are generally made from a process utilizing an apparatus that enables sufficiently high shear, temperature (above the gelling temperature) and residence time so as to provide a homogeneous molten mass of the composition and allow formation of the gel upon cooling. This is generally accomplished in the apparatus by heating, hydrating, mixing, solubilizing and optionally deaerating the composition. Examples of such an apparatus include Ross mixers, Stephan processors, extruders, jet cookers and the fluid mixing apparatus as set forth in Figure 1. Ross mixers, Stephan processors and conventional jet cookers are readily available commercially. Prior to cooling, the molten mass may optionally be fed to at least one of a pump, mixer or devolatilizer; e.g., s an extruder. Note, as an additional aspect of the invention, the molten mass need not necessarily reach homogeneity in step (i). That is, when an additional apparatus such as a mixer, pump and/or devolatilizer is used, homogeneity of the molten mass can be obtained prior to or after feeding the molten composition into at least one of the mixer, pump and/or devolatilizer provided the molten mass reaches homogeneity prior to gelling. An extruded molten mass can also be directed to a film forming or shaping device (e.g. spreader box) that aids in the uniform casting of a continuous film, or, through a die that allows a direct formation of a film or shaped extrudate from the molten mass delivery equipment. Care must be taken to maintain the molten mass above the initiation of restricted flow/gel structure formation. Insulated and pre-heated (to maintain proper temperatures) transfer hoses may be used to insure molten mass flow until desired gel film formation is initiated. Additional processing methods (such as pre-heating the discharge/plunger-like head as seen in a Ross process system) can force (by pressure) the molten mass through the transfer hoses mentioned above. Additional insulation can help maintain molten mass temperatures through the use of a Teflon disk initially placed upon the molten mass surface immediately after removing the mixing device. Note, as an additional aspect of the invention, the molten mass need not necessarily reach full homogeneity in step (i). That is, when an additional apparatus such as a mixer, pump and/or devolatilizer is used, homogeneity of the molten mass can be obtained prior to or after feeding the molten composition into at least one of the mixer, pump or devolatilizer provided the molten mass reaches homogeneity prior to gelling.

As used herein, a "fluid mixing apparatus" refers to the apparatus in FIG. 1. FIG. 1 illustrates a fluid mixing apparatus 10. The fluid mixing apparatus 10 is arranged to mix steam 2 with a first fluid or slurry 4 and a second fluid or slurry 6 to produce a molten mass or slurry mixture 8.

The fluid mixing apparatus 10 comprises a first housing 20 having a first inlet 22 through which the steam 2 enters the housing 22, a nozzle end 24 from which the steam 2 exits the housing 20, and a nozzle valve or stem 26 disposed at the nozzle end 24. An actuator means 30 is connected to the first housing 20 for controlling the exit rate or exit pressure of the first fluid 2 at the nozzle end 24. The actuator means 30 may be of the type manufactured by Fisher Controls U.S.A.

The fluid mixing apparatus 10 further comprises a second, mixing housing 40 coupled to the first housing 20 at the nozzle end 24 of the first housing 20. The second housing 40 includes a second inlet 42 through which the first fluid 4 enters the second housing 40, and a third inlet 44 through which the second fluid 6 enters the second housing 40. The inlets 42 and 44 are disposed downstream of the first inlet 22. As shown in FIG. 3, the second inlet 42 and third inlet 44 are disposed in a common plane and spaced apart radially from each other, most preferably directly opposite (i.e., 180º apart) about the central axis Y of the mixing apparatus 10. The second housing 40 defines a generally cylindrical mixing chamber 52 that in turn defines a flow passage extending along the axial length of the mixing chamber 52 from an entry end 54 of the mixing chamber 52 to an exit end 56 of the chamber 52. The nozzle valve 26 is movable by the actuator 30 between seated and unseated positions at the entry end 54 to control the flow rate of steam 2 into the mixing chamber 52.

The nozzle end 24 of the first housing 20 directs the steam 2 into the entry end 54 of the mixing chamber 52. The second inlet 42 and the third inlet 44 radially direct the first fluid 4 and second fluid 6, respectively, into the mixing chamber 52. The steam 2, first fluid 4 and second fluid 6 are mixed in the mixing chamber 52 to form a molten mass or mixture 8 which exits the mixing chamber 52. The molten mass 8 then may be shaped into a shaped article or formed into a film, such as by casting the mixture 8 onto a cooling drum or by passing the mixture 8 through an extruder.

The fluid mixing apparatus 10 is adapted to produce a mixture for forming a film, more particularly an edible film for making edible delivery systems. Incompatible film components generally are placed in different fluid inlet streams so that such incompatible components come together in the first instance at the interface of the steam injection within the mixing chamber 52 of the fluid mixing apparatus. While FIG. 1 shows inlets for steam, and first and second fluids, one or more additional inlets for one or more additional fluids may be provided. Preferably, the housings 20, 40 and other components of the fluid mixing apparatus 10 are constructed of high-grade stainless steel.

The gel film can also be used to modify the dissolution profile of the dosage forms. For example, gel films of the invention can contain added components that can create solid dosage forms having immediate release, enteric or delayed release capabilities. Definitions of "immediate release", "delayed release" and "enteric" can be found in the U.S. Pharmacopeia and are incorporated herein by reference.

The present invention is now described in more detail by reference to the following examples, but it should be understood that the invention is not construed as being limited thereto. Unless otherwise indicated herein, all parts, percents, ratios and the like are by weight.

### EXAMPLES

Unless otherwise indicated, the following procedures were used to prepare and evaluate the materials and films in Examples 1-3. The Stephan UMC5 processor is a laboratory scale mixing device that provided suitable high shear mixing, heating, and deaerating of the formulations which were cast as films in the laboratory. A suitable batch size used was 1500 grams.

An aqueous starch dispersion was prepared by dissolving any salts/buffers and pH modifiers in deionized water. The starch and/or maltodextrin (M100) were added and mixed until dissolved/dispersed. Pure Cote® B760 starch is available from the Grain Processing Corporation of Muscatine, Iowa. A hydrocolloid mixture was prepared in the Stephan UMC5 processor by premixing the plasticizers until uniform, and adding the preblended dry hydrocolloids portionwise while mixing for about 30 seconds at 200 rpm after each addition. Sorbitol Special and glycerin were used as plasticizers. Sorbitol Special is an aqueous solution of sorbitol and sorbitol anhydrides at 76% solids supplied by SPI Polyols, Inc. (New Castle, DE).

The starch dispersion was added to the non-aqueous hydrocolloid mixture and mixed at 300 rpm for 5 minutes. The mechanical agitation was increased to 2100 rpm and the mixture was heated to 85°C to 95°C with mixing. When the target temperature was achieved, the mixture was stirred for 30 minutes, then the sample was held under vacuum (50-60 bars) with continued agitation for an addition 45 minutes.

When the hold time under vacuum at temperature has been completed, the sample was poured into a preheated wide mouth quart Mason jar. Temperature and pH were recorded. Viscosity was measured on the hot sample using an Brookfield LVF viscometer.

A small portion of the sample was set aside and refrigerated usually overnight prior to measurement of gel/melt properties and solids using an Atago E series hand held refractometer (Gardco, Pompano Beach, FL). The melt temperature was determined by placing a small chunk of the refrigerated gel on a wire string stand held within a test tube so that the chunk does not contact the wall of the test tube. The test tube was covered with aluminum foil with a small hole to allow measurement of the gel temperature using a digital tempermeter probe. The test tube was immersed in the heating bath so that the chunk is below the surface of a hot water bath at approximately 100^{o}C. A silicone oil bath was used for samples that had melt temperatures above 90^{o}C. The melt temperature was recorded when the gelled sample became wet in appearance, softened and could be stirred (a temperature range was noted). Once the sample had melted, the test tube was transferred to a second beaker containing cold tap water (15^{o}C). The temperature probe was used to record the temperature as the sample was cooled and to probe the sample surface to determine whether the sample had begun to gel. The gel temperature was the temperature upon cooling where the sample no longer flowed to fill in an indentation made by the probe.

The hot sample was then cast, using a draw down bar with a gap set at 3 mm clearance, onto 177 mm by 177 mm by 5 mm metal plates which were pre-sprayed with PAM (lecithin) to facilitate easy removal of film material. The gel coated plates were covered to avoid loss of moisture on the cast film and refrigerated (less than 8^{o}C) for at least one-half hour prior to removal of the film for testing. Refrigeration is not required for film formation. Dried film strips were prepared by drying the coated plates in a 40°C forced air/fan oven. Films dried 2 hours at 40^{o}C gave an intermediate solids of about 60% while films dried overnight at 40^{o}C typically gave solids of 80% or higher. Test properties were measured at room temperature (about 20^{o}C) unless otherwise indicated. The percent of solids in the dried film was determined by difference in weight between the cast film using its formulated solids and the dried film. Break force strengths were measured on the cast and dried film strips using a Texture Analyzer TA-108S Mini Film Test Rig.

Unless otherwise indicated, Maltrin M100 was obtained from Grain Processing Corporation, Pure-Cote B760 was obtained from Grain Processing Corporation, Sorbitol Special was obtained from SPI Polyols and Glycerin was obtained from VWR (EP/USP grade).

Example 1

Table I lists compositions and properties of gel films prepared using blends of sodium alginates with low viscosity guar Edicol ULV 50 obtained commercially from Indian Gum Industries Ltd.. Protanal® LFR 5/60, Protanal® LF 20/40 and Protanal® SF 120 RB are sodium alginates available from FMC Corporation (Philadelphia, PA).

TABLE I

| Ingredient (grams) | Ex-1-1 | Ex 1-2 | Ex 1-3 |
|---|---|---|---|
| Water | 836.3 | 836.3 | 836.3 |
| LFR 5/60 | 40.5 | 0 | 0 |
| LF 20/40 | 0 | 40.5 | 0 |
| SF120 RB | 0 | 0 | 30 |
| GUAR ULV50 | 49.5 | 49.5 | 45 |
| Starch B760 | 220.8 | 220.8 | 220.8 |
| M100 | 0 | 0 | 15.0 |
| SORBITOL SP | 264.4 | 264.4 | 264.4 |
| GLYCERIN | 88.2 | 88.2 | 88.2 |
| TOTAL WEIGHT | 1500.0 | 1500.0 | 1500.0 |
| Temp, ° C.* | 90 | 94 | 93 |
| VISCOSITY, MPAS* | 31,650 | <50,000 | <50,000 |
| Gel, ° C. | 50 | RT | RT |
| Melt, ° C. | 70-71 | 95* | 87* |
| PH | 5.6 | 5.5 | 5.6 |
| CAST FILM | 40% | 40% | 40% |
| SOLIDS, EST | | | |
| BF (G) | <40 | 102 | 110 |
| DRIED FILM(2 hrs@40° C.) | 60% | 64% | 67% |
| SOLIDS, EST | | | |
| BF (G) | 617 | 1250 | 1126 |
| Dried film (16 hours, 40 C) | 80% | 80% | 94% |
| SOLIDS, EST | | | |
| AVG FILM THICKNESS | 0.53 | 0.89 | 0.51 |
| BF (G) | 4780 | 7701 | 10,850 |

* Temperature and viscosity of molten mass prior to casting

Increasing the molecular weight of the sodium alginate contributed to the overall film structure and resulted in increased film strength (Examples 1-1, 1-2 and 1-3). The melt temperatures above were measured by heating the gel films using an oil bath. When heated, they soften and could be stirred. When allowed to cool, the gel formed above room temperature.

Example 2

Two potassium alginates were used: KAHG was the potassium salt of alginic acid extracted from Laminaria hyperborean and contains a high level guluronic acid (G) units. KAHM was a potassium salt of alginic acid extracted from Lessonia nigrescens and has a high level of mannuronic (M) units. The viscosity of the KAHG and KAHM were 5 cps and 12 cps, respectively, when measured in a 1% solution of water at 25^{o}C.

Potassium cation associated with the alginate is beneficial for the initiation of the structuring of kappa carrageenan and/or kappa-2 carrageenan with the alginate into a homogeneous cast & set film. The kappa carrageenan was an alkali processed, clarified extract of Kappaphycus alaverei (Eucheuma cottonii). All hydrocolloids used had low levels of divalent cations as shown in Table II.

Table II: Cation Content of Hydrocolloids

| Test | | | |
|---|---|---|---|
| Viscosity | KAHG | KAHM | Kappa Cgn |
| Mg, % | 0.07 | 0.12 | 0.09 |
| Ca, % | 0 | 0 | 0.06 |
| K, % | 15.73 | 16.01 | 2.60 |
| Na, % | 0.63 | 0.59 | 5.45 |

Table III presents compositions and properties of films formed using mixtures of potassium alginates and other film formers such as kappa carrageenan and low viscosity guar gum available as Edicol ULV 50 from Indian Gum Industries.

TABLE III: FILMS USING LOW VISCOSITY GUAR, POTASSIUM ALGINATES AND CARRAGEENAN

| INGREDIENT | | | | | | |
|---|---|---|---|---|---|---|
| (G) | Ex 2-1 | Ex 2-2 | Ex 2-3 | Ex 2-4 | Ex 2-5 | Ex 2-6 |
| Water | 836.3 | 836.3 | 836.3 | 836.3 | 836.3 | 836.3 |
| KAHG | 60 | 60 | 40.5 | 10.5 | 0 | 0 |
| KAHM | 0 | 0 | 0 | 0 | 60.0 | 10.5 |
| Kappa Cgn | 30 | 30 | 0 | 30 | 30.0 | 30 |
| Guar | 0 | 0 | 49.5 | 49.5 | 0 | 49.5 |
| Starch B760 | 220.8 | 220.8 | 220.8 | 220.8 | 220.8 | 220.8 |
| SORBITOL SP | 264.4 | 264.4 | 264.4 | 264.4 | 264.4 | 264.4 |
| GLYCERIN | 88.2 | 88.2 | 88.2 | 88.2 | 88.2 | 88.2 |
| Total weight | 1500.0 | 1500.0 | 1500.0 | 1500.0 | 1500.0 | 1500.0 |
| Temp, ° C.* | 90 | 90 | 88 | 90 | 93 | 92 |
| VISCOSITY, | 26,500 | 28,650 | 24,800 | 28,250 | 42,650 | 31,250 |
| MPAS* | | | | | | |
| Gel, ° C. | 42 | 41 | 50-51 | 53 | 39 | 55 |
| Melt, ° C. | 60-65 | 62-67 | 60-61 | 70-74 | 60-63 | 65-69 |
| PH | 7.6 | 7.2 | 6.3 | 5.6 | 7.4 | 5.9 |
| Cast Film | 40% | 40% | 40% | 40% | 38% | 41% |
| Solids, est | | | | | | |
| BF (g) | <40 | <40 | <40 | 188 | <40 | 185 |
| Dried film(2 | 66% | 62% | 63% | 64% | 62% | 66% |
| hrs@40° C.) | | | | | | |
| Solids, est | | | | | | |
| BF (g) | 370 | 248 | 445 | 1811 | 502 | 1265 |
| Dried film | 81% | 79% | 85% | 80% | 77% | 80% |
| (16 hours, | | | | | | |
| 40 C) | | | | | | |
| Solids, est | | | | | | |
| Avg film | 0.83 | 0.76 | 0.56 | 0.60 | 0.56 | 0.59 |
| thickness (mm) | | | | | | |
| BF (g) | 3826 | 4253 | 4144 | 7960 | 6918 | 8301 |

* Temperature and viscosity of the molten mass prior to casting

Results indicate beneficial interaction by combining potassium alginate, either high guluronic or high mannuronic, or combinations thereof, with kappa carrageenan and guar. Further variation of weight ratios of alginate and second film formers and process variations can also be used to, again, either cast as a high solids (>80%) molten mass, formed, and cooled gel film as is or for further processing.

Example 3

Table IV lists compositions and properties of gel films prepared using propylene glycol alginate and kappa carrageenan. Protanal® ester BV4830 is a propylene glycol alginates available from FMC Corporation (Philadelphia, PA). HEC is hydroxyethylcellulose. Kappa carrageenan is described above in Example 2.

TABLE IV: PROPYLENE GLYCOL ALGINATE-BASED BLEND COMPOSITIONS

| Ingredient (g) | 3-1 | 3-2 | 3-3 |
|---|---|---|---|
| WATER | 840.3 | 836.3 | 840.3 |
| BV4830 | 91.2 | 49.5 | 66.0 |
| HEC | 1.9 | | |
| KAPPA CGN | 24.0 | 40.5 | 54.0 |
| K citrate | 2.9 | | |
| Starch B760 | 207.8 | 220.8 | 207.8 |
| SORBITOL SP | 264.4 | 264.4 | 248.8 |
| GLYCERIN | 88.2 | 88.2 | 83 |
| Temp, ° C.* | 91 | 87 | 89 |
| VISCOSITY, MPAS* | 24,800 | 6550 | 12500 |
| pH | 4.2 | 3.8 | 3.9 |
| Gel, ° C. | 59 | 42-43 | 43-44 |
| Melt, ° C. | 72-75 | 54-63 | 62-64 |
| Cast film | 45% | 38% | 36% |
| Solids, est. | | | |
| BF (g) | 136 | 89 | 113 |
| Dried@16 hours, 40° C. | 87% | 79.8 | 86.6 |
| Solids | | | |
| Avg film thickness (mm) | 0.72 | 0.68 | 0.79 |
| BF (g) | 8838 | 5244 | 7638 |

* TEMPERATURE AND VISCOSITY OF MOLTEN MASS PRIOR TO CASTING

Table V reports compositions and film properties for kappa-2 carrageenan used in combination with propylene glycol alginate and potassium alginate. The kappa-2 carrageenan was an alkali processed, clarified extract of a mixture of Gigartina skottsbergii and Sarcothalia crispata, primarily haploid (gametophyte) plants. About 10-20% (total) of lambda- and theta-carrageenans from diploid (tetrasporophyte) plants were also present.

TABLE V: ALGINATE FILMS WITH KAPPA-2 CARRAGEENAN

| Ingredient (g) | Ex 3-4 | Ex 3-5 |
|---|---|---|
| Water | 834.7 | 834.7 |
| Kappa-2 Cgn | 40.5 | 54.0 |
| KAHG | 31.5 | 36 |
| BV4830 | 18.0 | 36.0 |
| M-100 | 227.3 | 227.3 |
| SORBITOL SP | 272.3 | 272.2 |
| GLYCERIN | 90.8 | 90.8 |
| Temp, ° C.* | 87 | 84 |
| VISCOSITY, MPAS* | 4250 | 1050 |
| Solids | 40 | 37 |
| Melt, ° C. | 77-78 | 74-79 |
| Gel, ° C. | 54 | 52 |
| pH | 4.8 | 5.5 |
| Cast film | | |
| (est 40% solids) | | |
| BF (g) | 142 | 168 |
| DRIED FILM | | |
| (est 80% solids)16 hours, 40 C | | |
| Avg film thickness | 0.67 | 0.48 |
| BF (g) | 3409 | 4004 |

* TEMPERATURE AND VISCOSITY OF MOLTEN MASS PRIOR TO CASTING

In example 3-4, potassium cations are supplied by the potassium alginate. The potassium cations promote carrageenan double helix formation at a temperature that allows the carrageenan to form its gel film structure. In example 3-5, the additional strength and lower processing viscosity is due to the higher level of propylene glycol alginate.

Table VI presents compositions and gel films formed from low viscosity guar Edicol ULV 50 in combination with propylene glycol alginate and other hydrocolloids. Protanal® ester BV4830 and Protanal® ester SLF3 are propylene glycol alginates available from FMC Corporation (Philadelphia, PA) and Kibun, respectively. The SLF-3 has a lower molecular weight compared to the BV-4830. HEC is hydroethylcellulose.

TABLE VI: FILMS USING PROPYLENE GLYCOL ALGINATE WITH GUAR

| Ingredient (g) | Ex 3-6 | Ex 3-7 | Ex 3-8 | Ex 3-9 |
|---|---|---|---|---|
| Water | 840.3 | 840.3 | 840.3 | 836.3 |
| BV4830 | 0 | 0 | 91.2 | 12.0 |
| SLF-3 | 114 | 85.5 | 0 | 0 |
| HEC | 2.4 | 1.8 | 1.9 | 0 |
| Kappa cgn | 0 | 0 | 24 | 40.5 |
| GUAR ULV 50 | 0 | 30 | 0 | 37.5 |
| Starch B760 | 207.8 | 207.8 | 207.8 | 220.8 |
| M-100 | 0 | 0 | 0 | 0 |
| NA CITRATE | 3.6 | 2.7 | 0 | 0 |
| K CITRATE | 0 | 0 | 2.9 | 0 |
| KCL | 0 | 0 | 2.4 | 0 |
| SORBITOL SP | 248.8 | 248.8 | 248.8 | 264.4 |
| GLYCERIN | 83 | 83.0 | 83.0 | 88.2 |
| Temp, ° C.* | 91 | 87 | 90 | 90 |
| VISCOSITY, MPAS* | 3250 | 16,500 | 25,000 | 23,100 |
| Gel, ° C. | 34-35 | 34-38 | 43-46 | 46 |
| Melt, ° C. | 58-60 | 62-64 | 56-62 | 60-68 |
| PH | 4.4 | 4.5 | 4.3 | 4.6 |
| Cast Film | 39% | 41 | 45& | 41.5% |
| Solids, est | | | | |
| BF (g) | <40 | <40 | 231 | 147 |
| Dried film(2 hrs@40° C. | 74% | 65% | 55% | 60% |
| Solids, est | | | | |
| BF (g) | 1877 | 355 | 842 | 592 |
| Dried film (16 hours, 40 C) | 85% | 77% | 78% | 80% |
| Solids, est | | | | |
| Avg film thickness (mm) | 0.67 | 0.60 | 0.75 | 0.62 |
| BF (g) | 4677 | 3317 | 9599 | 7214 |

* Temperature and viscosity of molten mass prior to casting

Example 4

The following examples show films made using the fluid mixing apparatus of Figure 3. In these examples, Part A and Part B were pumped from separate holding tanks at ambient temperature, as two separate streams 4, 6, into two different inlet ports 42, 44 which fed the steam injection fluid mixing apparatus device 10. The two individual streams 4, 6 were combined at the interface of the steam in the mixing zone 52 of the fluid mixing apparatus 10. The separate solutions of Part A and Part B were readily pumped into the fluid mixing apparatus 10 and mixed with steam 2. The steam 2 was introduced to the mixing zone at a pressure of 120 psi. The resulting molten mass or slurry mixture 8 flowed out of the exit port 56 of the fluid mixing apparatus 10. The mixture 8 was poured onto a smooth surface and drawn down to form a homogeneous film 9.

To measure the viscosity of the mixture 8, approximately 500 ml sample of the mixture 8 was collected from the outlet 56 and poured into a jar. The temperature, pH and viscosity were measured for this sample at 95ºC. A Brookfield LVF viscometer was used to measure the viscosity. An appropriate speed and spindle combination were used such that a reading could be taken. The dial reading was converted to dynamic viscosity (cP).

To measure the film strength and solids level, the molten mass 8 was collected from the outlet 56 then cast using a draw down bar, with a gap set at 3 mm, onto a stainless steel metal plate. The initial films 9 or "fresh films" were collected. Portions of the fresh films 9 were dried by placing them in a 40ºC forced air oven. Break force was measured on the fresh and dried film strips using a Texture Analyzer TA-108S Mini Film Test Rig. The percent solids were determined by measuring the difference between the initial weight of the fresh film and the final weight of the dried films.

To measure the gel temperature, a portion of the molten mass 8 was collected from the outlet 56 of the mixing apparatus 10 and transferred to a test tube. Half of the test tube remained empty. A glass thermometer was inserted into the molten mass 8. The material 8 was allowed to cool under room temperature conditions. After each degree of cooling, the thermometer was removed from the material 8. When a small, temporary indentation was observed in the surface of the mass 8, this temperature was recorded. The thermometer was re-inserted into the mass 8, which was allowed to cool further. The thermometer was removed and re-inserted at every degree of cooling until such a time as a permanent indentation formed in the mass 8, such that the indentation did not refill. The temperature at which the permanent indentation formed was recorded. The gel temperature reported was the range between the two recorded temperatures.

Table VII - Mixtures Containing PGA

| | Example No. | | | | |
|---|---|---|---|---|---|
| | 4-1 | 4-2 | 4-3 | 4-4 | 4-5 |
| Part A (%) | | | | | |
| Carrageenan B | 2.7 | 3.2 | 3.2 | 4.0 | 0.0 |
| kappa-2 | | | | | |
| Carrageenan A | 0.0 | 0.0 | 0.0 | 0.0 | 4.0 |
| Kappa | | | | | |
| PGA | 3.3 | 3.9 | 3.9 | 4.9 | 4.9 |
| Glycerin | 22.4 | 26.5 | 26.5 | 33.5 | 33.5 |
| Part B (%) | | | | | |
| KOH | 0.0 | 0.0 | 0.1 | 0.0 | 0.0 |
| K_{2CO}₃ | 0.0 | 0.0 | 0.0 | 0.3 | 0.3 |
| Starch | 13.9 | 16.4 | 16.4 | 20.7 | 20.7 |
| Water | 57.8 | 50.0 | 49.9 | 36.6 | 36.6 |
| Mixing chamber | 108 | 107 | 108 | 107 | 108 |
| temp. (° C.) | | | | | |
| Cutlet temp (° C.) | 102 | 102 | 102 | 101 | 102 |
| Viscosity cP | 5500 | 4650 | 2200 | 12400 | 9400 |
| (@95° C.) | | | | | |
| PH | 4.1 | 4.2 | 8.7 | 6.3 | 6.8 |
| % solids | 42 | 50 | 50 | 65 | 65 |
| Gel temp. (° C.) | 35-40 | not tested | not tested | 58-66 | 63-71 |
| wet film strength | 60 | 117 | 3874 | 337 | 822 |
| (grams) | | | | | |
| dry film strength | 2408 | 3069 | 4335 | 4561 | 4795 |
| (grams) | | | | | |

Table VII shows that the film former can be a combination of hydrocolloids, such as carrageenan and PGA. Additionally, salts can be added to influence film properties such as strength, gel temperature and pH.

The following Tables VIII and IX provide further descriptions of the components specified in the above examples.

Table VIII - Component Descriptions

| | | | |
|---|---|---|---|
| Name | Trade Name | Supplier | Description |
| Propylene glycol | Protanal BV 4830 | FMC BioPolymer | |
| alginate (PGA) | | | |
| Glycerin | | Callahan Chemical | 99.7% |
| Starch | Pure-Cote B790 | Grain Processing | |
| | | Corporation | |

The following Table IX provides a description of the various carrageenans used in this example.

Table IX - Carrageenan Descriptions

| | | | |
|---|---|---|---|
| | Carrageenan | | |
| Reference | Type | Description | Supplier |
| Carrageenan | Kappa | An alkali processed, clarified | FMC |
| A | | kappa-carrageenan extract of | Corporation |
| | | Kappaphycus alverezil | |
| | | (Eucheuma cottonii) with | |
| | | low divalency | |
| Carrageenan | Kappa-2 | An alkali processed, clarified, | FMC |
| B | | low divalency extract of a | Corporation |
| | | mixture of Gigartina | |
| | | skottabergii and Sarcothalia | |
| | | crispata, primarily haploid | |
| | | (gametophyte) plants, such | |
| | | extract being commonly | |
| | | known as “kappa-2 | |
| | | carrageenan”. Includes about | |
| | | 10-20% (total) of lambda and | |
| | | theta-carrageenans from | |
| | | diploid (tetrasporophyte) | |
| | | plants. Defined as the natural | |
| | | random block copolymer of | |
| | | kappa- and iota-carrageenan | |
| | | in the ratio of about 1.0 to | |
| | | 3.0:1 respectively, and has | |
| | | significantly different | |
| | | functionalities compared to | |
| | | mixing individual kappa and | |
| | | iota-carrageenan natural | |
| | | polymers at the same ratio. | |

Example 5

Delivery films of the invention were prepared by the following method: alginate and kappa carrageenan were dry blended to form a gum blend. KAHG and NAHG were the potassium salt and the sodium salt, respectively, of alginic acid extracted from Laminaria hyperborean and contains a high level guluronic acid (G) units. KAHM was a potassium salt of alginic acid extracted from Lessonia nigrescens and has a high level of mannuronic (M) units. The 1% aqueous solutions of the KAHG and KAHM had a viscosity of 5 cP and 12 cP, respectively, when measured in a 1% solution of water at 25^{o}C. Maltrin M 100 maltodextrin (Grain Processing Corp., Muscatine, Iowa) was dry blended with the gum blend. Deionized water and glycerin were weighed into a 1.2 liter stainless steel beaker. The dry premix was added to the water with good agitation, then heated to 90 ^{o}C and held within the temperature range 90^{o}C to 95^{o}C for 15 minutes to fully hydrate the gums. After replacing any water loss due to evaporation, the delivery ingredient was added and mixed two minutes for dispersion. The delivery ingredients tested were: (1) natural and artificial strawberry flavor (Dragoco, 0.1%), (2) titanium dioxide, and (3) caffeine. The hot solution was quickly poured into containers. Solution cast in petri dishes was cooled to room temperature to form films, then dried overnight in a forced draft oven at 45 ^{o}C to a constant weight. A sample was cooled then refrigerated (less than 8^{o}C) overnight prior to measurement of gel/melt properties and solids using an Atago E series hand held refractometer (Gardco, Pompano Beach, FL). The melt temperature was determined by placing a small chunk of the refrigerated gel on a wire string stand held within a test tube so that the chunk did not contact the wall of the test tube. The test tube was covered with aluminum foil with a small hole to allow measurement of the gel temperature using a digital tempermeter probe. The test tube was immersed in the heating bath so that the chunk is below the surface of a hot water bath at approximately 100^{o}C. A silicone oil bath was used for samples that had melt temperatures above 90^{o}C. The melt temperature was recorded when the gelled sample became wet in appearance, softened and could be stirred (a temperature range was noted). Once the sample had melted, the test tube was transferred to a second beaker containing cold tap water (15^{o}C). The temperature probe was used to record the temperature as the sample was cooled and to probe the sample surface to determine whether the sample had begun to gel. The gel temperature was the temperature upon cooling where the sample no longer flowed to fill in an indentation made by the probe. Break force (BF) and penetration were measured on the cast and dried film strips using a Texture Analyzer TA-108S Mini Film Test Rig. Rigidity was calculated as the BreakForce divided by the penetration.

Table X: Alginate Delivery Systems

Ingredient (g)

| | | | | | | |
|---|---|---|---|---|---|---|
| NAHG | 1.65 | 0.00 | 1.65 | 0.00 | 0.00 | 0.00 |
| KAHM | 0.00 | 0.00 | 0.00 | 0.00 | 0.00 | 1.65 |
| Kappa carrageenan | 1.25 | 1.25 | 1.25 | 1.25 | 1.25 | 1.25 |
| Maltodextrin | 7.00 | 7.00 | 7.00 | 7.00 | 10.00 | 10.00 |
| Water | 80.00 | 80.00 | 80.00 | 80.00 | 80.00 | 80.00 |
| Glycerin | 6.70 | 6.70 | 6.70 | 6.70 | 6.70 | 6.70 |
| Flavor | 3.30 | 3.30 | 0.00 | 0.00 | 0.00 | 0.00 |
| TiO2 | 0.00 | 0.00 | 3.30 | 3.30 | 0.00 | 0.00 |
| Caffeine | 0.00 | 0.00 | 0.00 | 0.00 | 0.30 | 0.30 |
| As cast film | ˜22% | ˜22% | ˜22% | ˜22% | ˜22% | ˜22% |
| Solids | | | | | | |
| BF, (g) | 190 | 2075 | 197 | 1562 | 1986 | 1686 |
| Penetration, cm | 0.4 | 0.3 | 0.4 | 0.3 | 0.4 | 0.3 |
| Rigidity | 476 | 6916 | 492 | 5036 | 4966 | 5620 |
| Dried film | | | | | | |
| BF (g) | 8775 | 14549 | 8926 | 7551 | 11600 | 9453 |
| Penetration, cm | 1.3 | 1.1 | 1.7 | 1.2 | 1.3 | 1.1 |

While the invention has been described in detail and with reference to specific embodiments thereof, it will be apparent to one skilled in the art that various changes and modifications can be made therein without departing from the spirit and scope thereof.

1. A delivery system comprising a homogenous, thermoreversible gel film, wherein said gel film comprises: (i) a film forming amount of a water soluble thermoreversible alginate and optionally at least one of a plasticizer, a second film former, a bulking agent, and a pH controlling agent; and (ii) an active substance.

2. The delivery system of claim 1, wherein said active substance is at least one of an oral care agent, a breath freshening agent, a pharmaceutical agent, a nutraceutical agent, a salivary stimulant agent, cosmetic ingredient, agricultural active, a vitamin, a mineral, a coloring agent, a sweetener, a flavorant, a fragrance or a food.

3. The delivery system of claim 1, wherein said alginate is present in an amount of at least 0.25% by dry weight of the gel film.

4. The delivery system of claim 1, wherein said alginate is present in an amount of 0.25% to 25% by dry weight of the gel film.

5. The delivery system of claim 1, wherein said alginate is present in an amount of at least 10% of the total dry weight of film formers in the gel film.

6. The delivery system of claim 1, wherein said alginate is present in an amount of at least 40% of the total dry weight of film formers in the gel film.

7. The delivery system of claim 1, wherein said alginate is present in an amount of at least 60% of the total dry weight of film formers in the gel film.

8. The delivery system of claim 1, wherein said alginate is present in an amount of at least 80% of the total dry weight of film formers in the gel film.

9. The delivery system of claim 1, wherein said alginate is the only film former present in the gel film.

10. The delivery system of claim 1, wherein said second film former is selected from the group consisting of starch, starch derivative, starch hydrozylate, cellulose gums, kappa carrageenan, iota carrageenan, polymannan gums, dextrans, pullulan, gellan, pectin, alkylcellulose ethers and modified alkyl cellulose ethers.

11. The delivery system of claim 1, wherein said plasticizer is at least one member selected from the group consisting of glycerin, sorbitol, maltitol, lactitol, solubilized oil, and polyalkylene glycols; said second film former is at least one member selected from the group consisting of a starch, starch derivative, starch hydrozylate, cellulose gum, kappa carrageenan, iota carrageenan, polymannan gums, dextran, pullulan, gellan, pectin, an alkylcellulose ether and a modified alkyl cellulose ether; and said bulking agent is at least one member selected from the group consisting of microcrystalline cellulose, microcrystalline starch, starch, starch derivatives, inulin, and starch hydrozylates.

12. The delivery system of claim 1 having a break force strength of at least 2,500 grams.

13. The delivery system of claim 1 having a break force strength of at least 4,000 grams.

14. The delivery system of claim 1 having a break force strength of at least 5,000 grams.

15. The delivery system film of claim 1 having a break force strength of at least 6,000 grams.

16. A process for preparing the homogeneous gel film delivery system in any of claims 1-15 comprising the steps of:
(i)heating, hydrating, mixing, solubilizing and optionally de-aerating said alginate and optionally at least one of a plasticizer, a second film former, a bulking agent, and a pH controlling agent in an apparatus providing sufficient shear, temperature and residence time to form a homogeneous molten composition, wherein said temperature is at or above the solubilizing temperature of said composition;
(ii)adding an effective amount of an active substance either prior to or after formation of the molten composition; and
(iii)cooling said molten composition containing said active substance at or below its gelling temperature to form said gel films containing said active substance.

17. The process of claim 16, wherein said active substance is at least one of an oral care agent, a breath freshening agent, a pharmaceutical agent, a nutraceutical agent, a salivary stimulant agent, a vitamin, a mineral, a cosmetic ingredient, an agricultural active, a coloring agent, a sweetener, a flavorant, a fragrance, a food.

18. The process of claim 16, wherein said apparatus is a Ross mixer, extruder, Stephan processor, jet cooker or fluid mixing apparatus.

19. The delivery system of claim 1 having a break force strength of at least 250 grams.

20. The delivery system of claim 1 having a break force strength of at least 1,000 grams.

21. The delivery system of claim 1, further comprising a flavorant and having a solids content of at least 50%.

22. The delivery system of claim 1, wherein said second film former is a carrageenan having a viscosity of less than 10 cps at 75 °C when measured in a 0.10 molar aqueous sodium chloride solution containing 1.5% by weight of said carrageenan based on the weight of all components in said solution.

23. The delivery system of claim 1 wherein said gel film does not contain a plasticizer.

24. The delivery system of claim 1 consisting of said water soluble thermoreversible alginate, a bulking agent, an active substance and water.

25. The delivery system of claim 24, wherein said bulking agent is corn syrup.
